(19) United States
(12) Patent Application Publication
(10) Pub. No.: US 2026/0249277 A1
(43) Pub. Date: Aug. 27, 2026

(54) SHAPED CATALYST BODY

(71) Applicant: BASF CORPORATION, Florham Park, NJ (US)

(72) Inventors: Jian-Ping CHEN, Beachwood, OH (US); Huan WANG, Shanghai (CN); Yanxia LIU, Shanghai (CN); Mario SOORHOLTZ, Shanghai (CN); Michael P. BARAN, Richmond Heights, OH (US); Gary J. BUNTING, Erie, PA (US)

(73) Assignee: BASF CORPORATION, Florham Park, NJ (US)

(21) Appl. No.: 18/877,412

(22) PCT Filed: Jun. 21, 2023
(86) PCT No.: PCT/CN2023/101808

§ 371 Date: Dec. 20, 2024

(30) Foreign Application Priority Data
Jun. 22, 2022 (WO) PCT/CN2022/100447

(51) Int. Cl. B01J 23/889; B01J 35/30; B01J 35/32; B01J 35/61; B01J 35/63; B01J 35/64; B01J 35/69; B01J 37/00; B01J 37/02; B01J 37/03; B01J 37/08; C07C 29/149

(52) U.S. Cl. CPC B01J 23/8892; B01J 35/32; B01J 35/394; B01J 35/613; B01J 35/633; B01J 35/635; B01J 35/651; B01J 35/69; B01J 37/0009; B01J 37/009; B01J 37/0236; B01J 37/031; B01J 37/088; C07C 29/149

(57) ABSTRACT

The present invention relates to a shaped catalyst body containing copper, aluminum, and manganese, a process for producing the shaped catalyst body, and a process for using the shaped catalyst body for hydrogenation, dehydrogenation, hydrogenolysis, or ethynylation.

### FIELD OF THE INVENTION

The present invention relates to a shaped catalyst body containing copper, aluminum, and manganese, a process for producing the shaped catalyst body, and a process for using the shaped catalyst body for hydrogenation, dehydrogenation, hydrogenolysis, or ethynylation.

### BACKGROUND

In recent years, fatty alcohol produced by the hydrogenolysis of carboxylic esters shows significant growth of demand due to hygiene request against the coronavirus pandemic. Heterogeneous catalysts, including noble metal, Nickle, Cobalt and Copper, are widely used in this reaction. Commercial catalysts for hydrogenolysis of fatty acid esters commonly utilize Copper-Chrome (Cu—Cr) composite, which have high performance and mechanical stability. Environmental issues involving disposal of Cr-containing catalysts, however, are expected to eventually eliminate their use in many countries. Therefore, it is more advantageous and sustainable to employ Cr-free Cu-containing catalysts having good catalyst activity to replace currently used Cu—Cr catalysts in hydrogenolysis of carboxylic esters. Pellet-shaped and extrudate-shaped CuZn—, CuMn— or CuMnAl— containing material can be used as active catalyst for hydrogenolysis of carboxylic esters.

The U.S. patent Ser. No. 10/226,760 B2 describes a tableted Cu—Zn catalyst starting from thermally treated metal carbonate mixture produced by precipitation approach. It has been found that the carbonate content correlates with the Cu metal surface area of the reduced catalysts. The catalysts are employed for hydrogenolysis of a C12-methyl ester feed at a temperature of 180° C. under a pressure of 280 bar in Examples. Their invented catalysts display a significantly increased conversion of C12-methyl ester compared to the comparative catalyst produced with lower carbonate content.

The U.S. patent Ser. No. 10/315,188 B2 discloses a CuMnAl tableted catalyst body obtained by a process with addition of graphite material with specific particle diameter 5.0 μm≤D90≤17.5 μm. Their invented catalysts are employed for hydrogenation of a C12-methyl ester feed at a temperature of 180° C. under a pressure of 280 bar in Examples. It has been found that addition of graphite with smaller particle size and larger surface area can lead to an increase of ester conversion and decrease of paraffins by-product selectivity.

The U.S. patent Ser. No. 10/434,500 B2 describes a CuAl tableted catalyst body obtained by mixture of calcined and uncalcined carbonate produced by precipitation approach. Their invented catalyst has a particular bimodal porosity, wherein pores having a pore size in the range from 500 to 2500 nm accounts for ~13% of pore volume and pores having a pore size in the range from 5 to 45 nm accounts for ~75% of pore volume. But the pore volume formed by pores with pore size in the range from 45 to 200 nm is less than 10%. It has been found pore volume of catalyst after tableting, calcination and reduction varies as a function of uncalcined carbonate content. Their invented catalysts are measured for hydrogenolysis of a C12-methyl ester feed at temperatures of 160° C., 180° C. and 240° C. under a pressure of 280 bar in activity measurement Examples. It has been observed that catalyst with higher pore volume induced by uncalcined carbonate can lead to an increased conversion of fatty acid ester than comparative catalyst under all three selected temperatures.

In addition to above prior arts regarding Cr-free Cu catalyst tablets for hydrogenolysis of fatty acid esters, catalysts in extrudate-shape are also disclosed for this application. Generally, catalyst extrudates have a substantially higher pore volume and lower bulk density, while maintaining at least comparable mechanical strength compared to catalyst tablets. The U.S. patent Ser. No. 10/639,616 B2 describes a catalyst extrudate body compromising 20~43 wt % of Cu, 20~40 wt % Al and 1~10 wt % Mn based on the total weight of the catalyst, wherein larger than 50% of the pore volume is formed by the pores having a pore size in the range from 7 to 40 nm. But the pore volume formed by pores with pore size in the range from 45 to 200 nm is less than 10%. It has been observed that their invented extrudate catalyst have higher pore volume and lower bulk density than comparative catalyst in form of tablets. Their invented catalysts are conducted for hydrogenolysis of a C12-methyl ester feed at temperatures of 160° C., 180° C. and 240° C. under a pressure of 280 bar. The data reveals a significant improvement in productivity to the target product has been achieved.

In the commercial use of catalysts in hydrogenolysis of methyl esters, as disclosed in above prior arts, are conducted in a liquid-phase process, which are typically operated under higher pressure range to facilitate ester conversion and alcohol selectivity. This leads to higher requirement for reactor design and control difficulty as well as higher cost. If the reaction can be conducted in lower pressures and reach similar productivity, it would lead to a considerable cost saving and lower risk for plant operation. In addition, a pressure decrease would be beneficial to longer operating life of the catalyst, especially when the catalyst is not sufficiently stable regarding to mechanical strength.

In view of this background, it is an object of the present invention to provide a Cr-free Cu catalyst with improved catalytic performance for hydrogenation, dehydrogenation, hydrogenolysis, or ethynylation at lower pressures and temperatures.

### SUMMARY OF THE INVENTION

It has been surprisingly found that the object was achieved by a shaped catalyst body with a substantially lower packed bulk density and pore volume in specific range comparing to the conventionally shaped catalysts.

Accordingly, in one aspect, the present invention relates to a shaped catalyst body containing copper, aluminum, and manganese, wherein the shaped catalyst body has a packed bulk density of 0.87 to 1.43 g/cc.

In another aspect, the present invention relates to a process for producing the shaped catalyst body comprising:
a) precipitation by combining aqueous solution of copper compound, manganese compound, aluminum compound and a precipitation agent;
b) filtration of the slurry and washing the precipitate;
c) drying and calcining at a temperature in the range from 200 to 1000° C. to form a thermally treated intermediate;
d) mixing the thermally treated intermediate; and
e) shaping to a shaped body.

In a further aspect, the present invention relates to a process for using the shaped catalyst body for hydrogenation, dehydrogenation, hydrogenolysis, or ethynylation.

### BRIEF DESCRIPTION OF THE DRAWINGS

FIG. 1 shows the pore size distribution of Example 1.

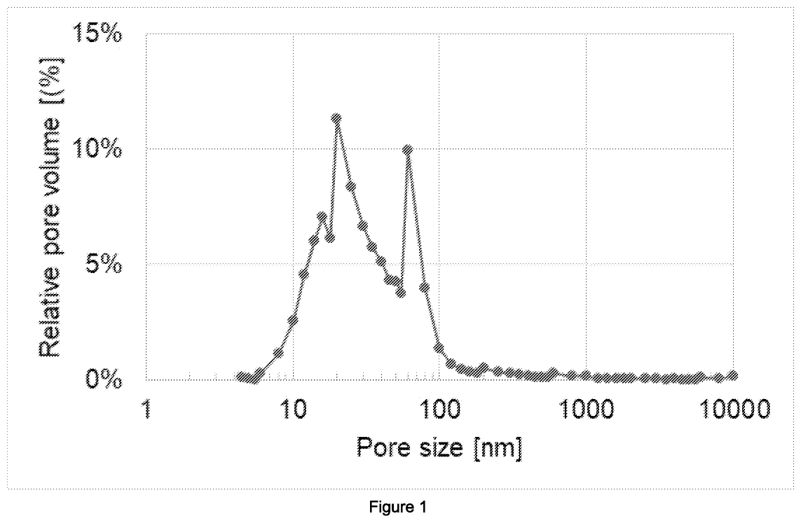

FIG. 2 shows the pore size distribution of Example 2.

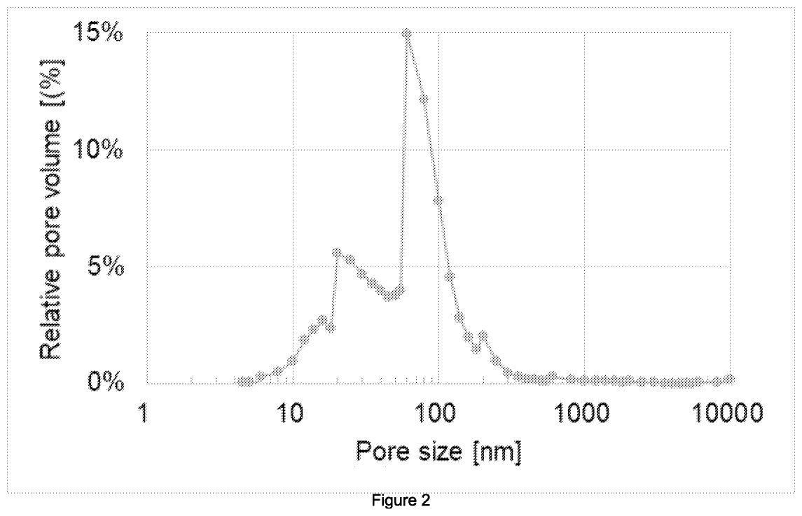

FIG. 3 shows the pore size distribution of Example 3.

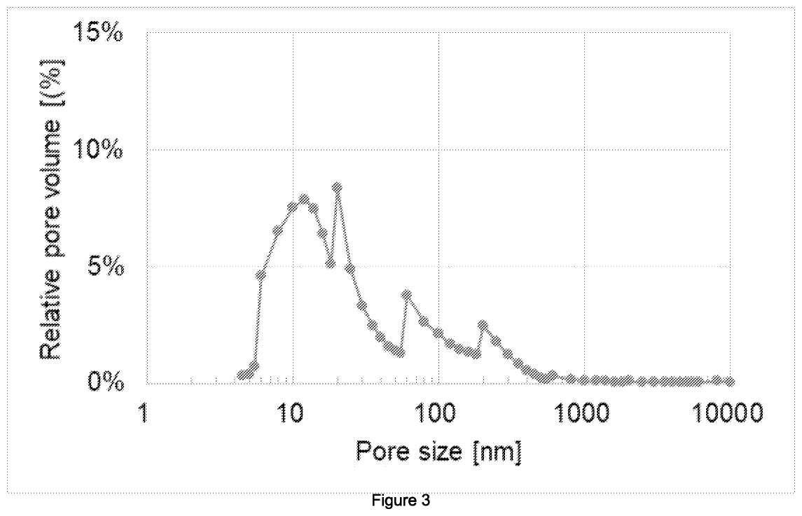

FIG. 4 shows the pore size distribution of Comparative Example 1.

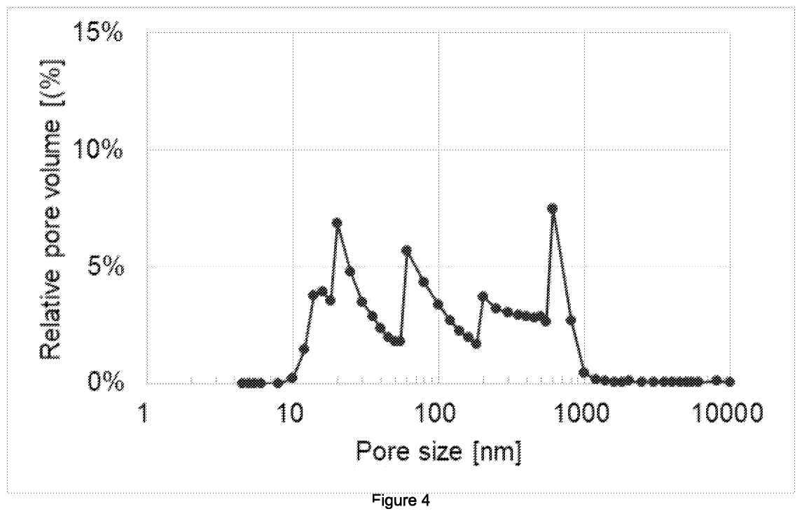

FIG. 5 shows the pore size distribution of Comparative Example 2.

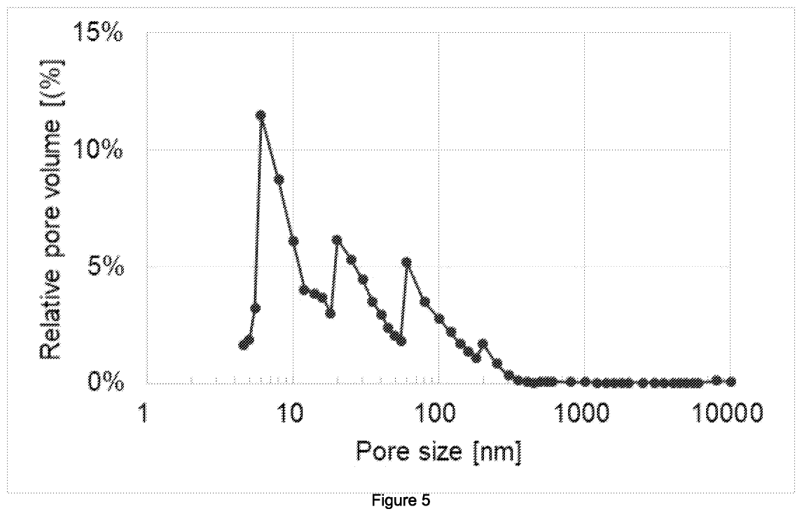

### DETAILED DESCRIPTION OF THE INVENTION

Before describing several exemplary embodiments of the invention, it is to be understood that the invention is not limited to the details of construction or process steps set forth in the following description. The invention is capable of other embodiments and of being practiced or being carried out in various ways.

With respect to the terms used in this disclosure, the following definitions are provided.

Throughout the description, including the claims, the terms “comprise”, “comprising”, etc. are used interchangeably with “contain”, “containing”, etc. and are to be interpreted in a non-limiting, open manner. That is, e.g., further components or elements may be present. The expressions “consists of” or “consists essentially of” or cognates may be embraced within “comprises” or cognates.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The terms “a”, “an” and “the” are used to refer to one or to more than one (i.e., to at least one) of the grammatical object of the article.

The term “and/or” includes the meanings “and”, “or” and also all the other possible combinations of the elements connected to this term.

According to the first aspect, the present invention provides a shaped catalyst body containing copper, aluminum, and manganese, wherein the shaped catalyst body has a packed bulk density of 0.87 to 1.43 g/cc, preferably 0.90 to 1.42 g/cc, more preferably 0.95 to 1.35 g/cc. For example, the packed bulk density includes, but is not limited to, about 0.87 g/cc, about 0.90 g/cc, about 0.93 g/cc, about 0.95 g/cc, about 1.0 g/cc, about 1.10 g/cc, about 1.20 g/cc, about 1.30 g/cc, about 1.35 g/cc, about 1.40 g/cc, about 1.42 g/cc, about 1.43 g/cc, or any range including and/or in between any two of the preceding values.

All references to packed bulk density in the specification and claims of the present invention are based upon measurements utilizing the method described in ASTM-D4 164-03.

In some embodiments according to the present invention, the shaped catalyst body comprises from 30% to 75%, preferably from 40% to 65%, more preferably 45% to 60% by weight of Cu, calculated as CuO. For example, the amount of the copper oxide may include, but is not limited to, about 30 wt %, 35 wt %, about 40 wt %, about 45 wt %, about 50 wt %, about 55 wt %, about 60 wt %, about 65 wt %, about 70 wt %, about 75 wt %, or any range including and/or in between any two of the preceding values.

It will be understood that the copper oxide and, when present, the at least one oxide of other metal (or element) may be present in form of respective oxides, or a composite oxide of copper and the other metal (or element), or a combination thereof.

In some embodiments according to the present invention, the shaped catalyst body comprises from 10% to 50%, preferably from 20% to 40%, more preferably 25% to 35% by weight of Al, calculated as Al₂O₃. For example, the aluminum oxide may be present in an amount of about 10 wt %, 15 wt %, about 20 wt %, about 25 wt %, about 30 wt %, about 35 wt %, about 40 wt %, about 45 wt %, about 50 wt % or any range including and/or in between any two of the preceding values.

It will be understood that the aluminum oxide and, when present, the at least one oxide of other metal (or element) may be present in form of respective oxides, or a composite oxide of aluminum and the other metal (or element), or a combination thereof.

In some embodiments according to the present invention, the shaped catalyst body comprises from 1% to 25%, preferably from 5% to 20%, more preferably 7% to 15% by weight of Mn, calculated as MnO₂. For example, the manganese oxide may be present in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, or any range including and/or in between any two of the preceding values.

It will be understood that the manganese oxide and, when present, the at least one oxide of other metal (or element) may be present in form of respective oxides, or a composite oxide of manganese and the other metal (or element), or a combination thereof.

In some embodiments according to the present invention, the shaped catalyst body may include a binder, where the binder includes but not limited to calcium silicate, sodium silicate, silica sol, clay, boehmite, and mixtures thereof.

In some embodiments according to the present invention, the bind64 includes a zirconium component. In any embodiment herein, the zirconium component may be present in the reduced metal or oxide forms or as a precursor to such forms and in one or more oxidation states as discussed above. For example, the zirconium component is present in the form of zirconium oxide. In any embodiment herein, the zirconium component is present in an amount from about 3 wt % to about 20 wt % by weight of Zr, calculated as ZrO₂. Suitable amounts of the zirconium component include, but are not limited to, from about 5 wt % to about 15 wt %, about 5 wt % to about 12 wt %, about 5 wt % to about 8 wt %, or any range including and/or in between any two of the preceding values. For example, the zirconium component may be present in an amount of about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt % or any range including and/or in between any two of the preceding values.

The shaped catalyst body as described herein in any embodiment may further include an alkali metal component. In any embodiment herein, the alkali metal is selected from the group consisting of sodium (Na), potassium (K), rubidium (Rb), cesium (Cs), and combinations thereof. These metals may be present in the reduced metal or oxide forms or as precursors to such forms and in one or more oxidation states as discussed above. For example, the alkali metal component may include sodium in the form of disodium oxide. In any embodiment herein, the alkali metal may be present in an amount from about 0 wt % to about 1 wt % by weight of the shaped catalyst body. For example, the alkali metal component may be present in an amount of about 0.01 wt %, 0.05 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, or any range including and/or in between any two of the preceding values.

In some embodiments according to the present invention, the shaped catalyst body has a bimodal pore size distribution.

All references to pore diameters and pore volumes in the specification and claims of the present invention are based upon measurements utilizing mercury intrusion method described in ASTM-D4284-03.

In specific embodiments according to the present invention, the shaped catalyst body exhibits a pore volume of 0.02 to 0.50 ml/g, preferably 0.15 to 0.30 by pores with pore size in the range from 45 to 200 nm, For example, the pore volume includes, but is not limited to, about 0.02 ml/g, about 0.05 ml/g, about 0.10 ml/g, about 0.15 ml/g, about 0.18 ml/g, about 0.20 ml/g, about 0.22 ml/g, about 0.25 ml/g, about 0.27 ml/g, about 0.30 ml/g, or any range including and/or in between any two of the preceding values; and a pore volume of 0.20 to 0.60 ml/g, preferably 0.25 to 0.50 by pores with pore size in the range from 10 to 200 nm. For example, the pore volume includes, but is not limited to, about 0.20 ml/g, about 0.25 ml/g, about 0.30 ml/g, about 0.35 ml/g, about 0.40 ml/g, about 0.45 ml/g, about 0.50 ml/g, about 0.55 ml/g, about 0.60 ml/g, or any range including and/or in between any two of the preceding values.

In other specific embodiments according to the present invention, the shaped catalyst body, from 10% to 80%, preferably from 35% to 70%, more preferably 45% to 65% of the pore volume is formed by pores with pore size in the range from 45 to 200 nm. For example, the percentage includes, but is not limited to, about 10%, about 15%, about 20% ml/g, about 25%, about 30%, about 35%, about 40%, about 45%, about 50%, about 55%, about 60%, about 65%, about 70%, about 75%, about 80%, or any range including and/or in between any two of the preceding values; and from 70% to 100% of the pore volume is formed by pores with pore size in the range from 10 to 200 nm. For example, the percentage includes, but is not limited to, about 70%, about 75%, about 80%, about 85%, about 90%, about 95%, about 100%, or any range including and/or in between any two of the preceding values.

In some embodiments according to the present invention, the shaped catalyst body catalyst may have a BET surface area of about 15 m²/g to about 70 m²/g. For example, the calcined shaped catalyst body has a BET surface area of about 15 m²/g, about 20 m²/g, about 25 m²/g, about 30 m²/g, about 35 m²/g, about 40 m²/g, about 45 m²/g, about 50 m²/g, about 55 m²/g, about 60 m²/g, about 65 m²/g, about 70 m²/g, or any range including and/or in between any two of the preceding values. In any embodiment herein, the calcined hydrogenation catalyst has a BET surface area of about 15 m²/g to about 70 m²/g, about 25 m²/g to about 65 m²/g, about 45 m²/g to about 60 m²/g, about 50 m²/g to about 60 m²/g, or any range including and/or in between any two of the preceding values.

According to the second aspect, the present invention provides a process for producing a shaped catalyst body comprising:
a) precipitation by combining aqueous solution of copper compound, manganese compound, aluminum compound and a precipitation agent;
b) filtration of the slurry and washing the precipitate;
c) drying and calcining at a temperature in the range from 200 to 1000° C. to form a thermally treated intermediate;
d) mixing the thermally treated intermediate; and
e) shaping to a shaped body.

The shaped catalyst body may be provided as tablets or extrudates. One way to process the blend of all of the ingredients is to extrude it through a shaping orifice to form an extruded catalyst body, or extrudate. Other catalyst bodies may be shaped into spheres or any other convenient formation. Another way is to tablet the catalysts. The shaped catalyst has a size from 1/32″ to 8 mm. For example, the hydrogenolysis catalyst may be extruded or tableted in sizes including, but not limited to, ⅛″ by ⅛″, 3/16″ by 3/16″, ¼″ by ¼″, 3/16″ by ¼″, ¼″ by 1/16″, or ⅛″ by 1/16″.

In some embodiments according to the present invention, the shaped catalyst body may be calcined. In any embodiment herein, the catalyst is a calcined and tableted catalyst.

The process includes calcining the material mixture at a temperature, and for a time, sufficient to cure form a calcined hydrogenolysis catalyst. In any embodiment herein, the calcining may occur at a temperature from about 200° C. to about 1000° C. For example, the calcining may occur at a temperature of about 200° C., about 250° C., about 300° C., about 350° C., about 400° C., about 450° C., about 500° C., about 550° C., about 600° C., about 650° C., about 700° C., about 750° C., about 800° C., about 850° C., about 900° C., about 950° C., about 1000° C., or any range including and/or in between any two of the preceding values. In any embodiment herein, the calcining temperature may be from about 300° C. to about 800° C., from about 400° C. to about 750° C., or from about 500° C. to about 700° C. In any embodiment herein, the calcination may occur over a period from about 0.5 h to about 4 h. In any embodiment, the calcination may occur over a period of about 0.5 h, about 1 h, about 1.5 h, about 2 h, about 2.5 h, about 3 h, about 3.5 h, about 4 h, or any range including and/or in between any two of the preceding values.

According to another aspect, the present invention provides a process for using the shaped catalyst body according to the present invention for hydrogenation, dehydrogenation, hydrogenolysis, or ethynylation.

The shaped catalyst bodies of the invention are suitable for use in numerous hydrogenation reactions. Preferably, the shaped catalyst in the invention is suitable for the liquid-phase hydrogenolysis of carboxylic acids and esters, preferably of fatty acids methyl ester mixtures having from 5 to 24 carbon atoms, to form the corresponding fatty alcohols. In particularly, the hydrogenolysis reaction of fatty acid methyl ester is suitable for operating under a specific pressure in the range from 60 to 250 bar, more preferably in the range from 75 to 100 bar.

Various embodiments are described hereinafter. It should be noted that the specific embodiments are not intended as an exhaustive description or as a limitation to the broader aspects discussed herein.

### EXAMPLES

### Example 1

Weigh out 122.2 g of Cu(NO3)2 (15.5 wt % Cu) solution and mix with Mn(NO3)2 solution with a molar ratio of Cu/Mn as 5.2/1.0. Weigh out NaAlO2 solution (12.5 wt % Al) with a molar ratio of Cu/Al as 1.2/1.0 and dilute with 18.9 g of deionized H2O. Add 450 ml of deionized H2O into a vessel. Weigh out 90.7 g of Na2CO3 powder and dissolve in deionized H2O to 500 ml. Simultaneously add Cu(NO3)2, Mn(NO3)2, NaAlO2 and Na2CO3 solution into the 450 ml deionized H2O. Keep the slurry during precipitation at a constant neutral pH. The precipitation temperature is held constant at room temperature. Afterwards, the precipitate is filtered, washed, and dried. The dried material is calcined in air at 600° C. to obtain the calcined metal carbonate material.

Above calcined metal carbonate powder is mixed with graphite powder. Afterwards, the mixture is formed into granules via a briquetting step and the granules are then tableted to form a shaped body. The tablets are then calcined at 750° C. The described material has a bulk density of 1.4 g/ml, pore volume of 0.26 ml/g and BET surface area of 53 m²/g. 93% of the pore volume is formed by pores with pore size in the range from 10 to 200 nm and specifically, 30% of the pore volume is formed by pores with pore size in the range from 45 to 200 nm.

### Example 2

Calcined metal carbonate powder material in Example 1 is mixed with graphite powder. Afterwards via briquetting step, the mixture is formed into granules with a specific bulk density, which is 40%-50% of the value of corresponding tablet product. The specific granules are then tableted to form a shaped body. The tablets are then calcined at 750° C. The described material has a bulk density of 1.1 g/ml, pore volume of 0.41 ml/g and BET surface area of 50 m²/g. 93% of the pore volume is formed by pores with pore size in the range from 10 to 200 nm and specifically, 59% of the pore volume is formed by pores with pore size in the range from 45 to 200 nm.

### Example 3

Calcined metal carbonate powder material in Example 1 is mixed with zirconium acetate, alumina, organic binder, water and then kneaded to form a wet mixture with the composition of 52 wt % CuO, 10 wt % MnO2, 30 wt % Al₂O₃ and 8 wt % ZrO2. The mixture was then extruded with an extruder to form a shaped body. The extrudates were then calcined at 500° C. The described material has a bulk density of 1.0 g/ml, pore volume of 0.31 ml/g and BET surface area of 50 m²/g. 76% of the pore volume is formed by pores with pore size in the range from 10 to 200 nm and specifically, 21% of the pore volume is formed by pores with pore size in the range from 45 to 200 nm.

### Comparative Example 1

Metal carbonate powder material is produced by the approach described in Example 1. Afterwards, the carbonate material is calcined in air at 800° C. and then mixed with calcium hydroxide, attapulgite, plasticizer, silica gel, water and then kneaded to form a wet mixture with the composition of 40 wt % CuO, 10 wt % MnO2, 20 wt % Al₂O₃, 20 wt % SiO2 and 10 wt % CaO. The mixture was then extruded with an extruder to form a shaped body. The extrudates were then calcined at 500° C. It has a bulk density of 0.8 g/ml, pore volume of 0.38 ml/g and BET surface area of 45 m²/g.

### Comparative Example 2

500 g of CuO powder is mixed with calcium hydroxide, plasticizer, silica gel, hydroxypropyl methylcellulose, water and then kneaded to form a wet mixture with the composition of 58 wt % CuO, 21 wt % SiO2 and 14 wt % CaO. The mixture was then extruded with an extruder to form a shaped body. The extrudates were then calcined at 500° C. The described material has a bulk density of 0.8 g/ml and BET surface area of 50 m²/g.

### Comparative Example 3

Weigh out 591 g of Cu(NO3)2 (15.5 wt % Cu) solution and mix with Al(NO3)3 solution (4.2 wt % Al) with a molar ratio of Al/Cu as 1.56/1.0 and 170 g of deionized H2O. Weigh out 442 g of Na2CO3 powder and dissolve with 1770 g of deionized H2O. Simultaneously add Cu(NO3)2, Al(NO3)3 and Na2CO3 solution into a separate vessel. Keep the slurry during precipitation at a constant pH about 6.0. The precipitation temperature is held constant at 80° C. Afterward, the precipitate is aged, filtered, washed, and dried. 507 g of the described dried powder, 87 g of pseudo-boehmite, 21 ml of formic acid and 375 g of water were mixed and kneaded to form a wet mixture. The mixture was then extruded to form an extrudate material, followed by calcination at 600° C. The described material has a bulk density of 0.85 g/ml and BET surface area of 21 m²/g.

### Comparative Example 4

Weigh out 320 g of Zn(NO3)2*6H2O and 336.4 g of Al(NO3)3*9H2O in 600 ml of deionized water. Weigh out 300 g of Na2CO3 powder and dissolve with 1200 g of deionized water. The two solutions are simultaneously added into a separated vessel with controlled neutral pH at a temperature of 50° C. Afterwards, the precipitate is aged, filtered, washed, dried and calcined at 400° C.

Above calcined powder is partially re-dissolved in a mixture solution of HNO3, Cu(NO3)2 and Zn(NO3)2 (atomic ratio of Cu:Zn=65:13) to form a suspension which has a total atomic ratio of Cu:Zn:Al=65:25:10. This suspension and 20 wt % of Na2CO3 solution are simultaneously added into a separated vessel with controlled pH of 6.8 at a temperature of 70° C. Afterwards, the precipitate is aged, filtered, washed, dried and calcined at 300° C.

Tablets are made from the powders after the powder is mixed with graphite powder, slugged and granulated. Then the granules are pressed into a tableted catalyst body. The described material has a bulk density of 1.45 g/ml and pore volume of 0.20 ml/g.

TABLE 1
Properties including compositions^{[}*^{]}, packed bulk density, pore volume and
relative pore volume in specific pore size range of inventive shaped catalysts

| | | | | | | | | | | | Relative | | |
|---|---|---|---|---|---|---|---|---|---|---|---|---|---|
| | | | | | | | | Packed | | Pore | pore | Pore | Relative |
| | | | | | | | | bulk | Pore | volume | volume | volume | pore volume |
| | CuO | MnO2 | Al2O3 | ZnO | SiO2 | CaO | ZrO2 | density | Volume | (45~200 nm) | (45~200 nm) | (10~200 nm) | (10~200 nm) |
| Sample | [%] | [%] | [%] | [%] | [%] | [%] | [%] | [g/ml] | [ml/g] | [ml/g] | (%) | [ml/g] | (%) |
| Example 1 | 58 | 12 | 30 | / | / | / | / | 1.4 | 0.26 | 0.08 | 30% | 0.24 | 93% |
| Example 2 | 58 | 12 | 30 | / | / | / | / | 1.1 | 0.41 | 0.24 | 59% | 0.38 | 93% |
| Example 3 | 52 | 10 | 30 | / | / | / | 8 | 1 | 0.31 | 0.07 | 21% | 0.24 | 76% |
| Comparative | 40 | 10 | 20 | / | 20 | 10 | / | 0.8 | 0.38 | 0.12 | 31% | 0.24 | 64% |
| Example 1 | | | | | | | | | | | | | |
| Comparative | 60 | / | 1 | / | 23 | 16 | / | 0.8 | / | / | / | / | / |
| Example 2 | | | | | | | | | | | | | |
| Comparative | 50 | / | 50 | / | / | / | / | 0.85 | / | / | / | / | / |
| Example 3 | | | | | | | | | | | | | |
| Comparative | 70 | / | 5 | 25 | / | / | / | 1.45 | 0.2 | 0.05 | 26% | 0.14 | 69% |
| Example 4 | | | | | | | | | | | | | |

^{[}*^{]}Composition values are calculated on oxide basis and normalized to 100%.

It can be seen from Table 1 that the invented catalysts show higher fraction of pore volume formed by pore size in the range from 10 to 200 nm than Comparative Examples. Specifically, pore volume formed by pore size in the range from 45 to 200 nm is even higher for invented catalyst than Comparative Examples.

### Hydrogenolysis of Fatty Acid Methyl Ester (FAME)

The activity of catalysts was tested in a multi-channel fixed bed reactor as follows. Each catalyst bed was formed from 2.5 ml of the catalyst tablets or extrudates in an electrically heated tubular reactor, supplied with hydrogen and nitrogen gas feed and means to feed liquid of C12-C18 methyl ester feedstock to the top of the catalyst bed. The catalyst was firstly activated by the well-known method in this industry. When the activation procedure was finished, the temperature and pressure was then adjusted to the desired reaction temperature and pressure and allowed to equilibrate under hydrogen. The reaction was begun by starting the feed of methyl ester and hydrogen. The product samples were taken after the reaction which had been allowed to equilibrate for 8 hours at each set of reaction conditions. Samples of the feedstock and of the product were analyzed by gas chromatography to evaluate the conversion of ester and the selectivity to hydrocarbon byproducts.
Temperature range: 170~250° C.
Pressure range: 75~250 bar
LHSV range: 0.3~1.5 h⁻¹
H2/feedstock molar ratio: (50~100)/1

Table 2 to Table 3 show the values of ester conversion and selectivity to hydrocarbons obtained at different temperature and pressure conditions.

The shaped catalysts with low packed bulk density, higher pore volume and specific pore size distribution produced according to the invention has a higher conversion of methyl ester and similar selectivities to hydrocarbon byproducts than comparative examples. This conversion difference is more significant at selected medium reaction pressures of 75 bar, 100 bar and selected medium temperatures of 170° C., 190° C. This can therefore be said that the plant operation with invented catalyst would lead to a considerable cost saving and lower risk because of mild operation temperature and pressure range.

TABLE 2
Ester conversion of examples and comparative examples

| | Ester Conversion (%) | | | | |
|---|---|---|---|---|---|
| | 170° C., | 170° C., | 170° C., | 190° C., | 250° C., |
| Sample | 75 bar | 100 bar | 250 bar | 75 bar | 75 bar |
| Example 1 | 66.0 | 75.0 | 90.0 | 95.0 | 99.8 |
| Example 2 | 83.3 | 88.0 | 97.0 | 99.0 | 99.9 |
| Example 3 | 65.0 | 72.0 | 92.0 | 98.5 | 99.9 |
| Comparative | 21.0 | 29.0 | 55.0 | 92.0 | 99.8 |
| Example 1 | | | | | |
| Comparative | 41.0 | 52.0 | 97.0 | 97.0 | 99.8 |
| Example 2 | | | | | |
| Comparative | 45.0 | 55.0 | 92.0 | 99.5 | 99.8 |
| Example 3 | | | | | |
| Comparative | 22.0 | 26.0 | 39.0 | 72.0 | 98.0 |
| Example 4 | | | | | |

TABLE 3
Selectivity to hydrocarbons of examples and comparative examples

| | Selectivity to hydrocarbons (%) | | | |
|---|---|---|---|---|
| | 170° C., | 170° C., | 170° C., | 190° C., |
| Sample | 75 bar | 100 bar | 250 bar | 75 bar |
| Example 1 | 0.02 | 0.02 | 0.02 | 0.05 |
| Example 2 | 0.03 | 0.03 | 0.02 | 0.07 |
| Example 3 | 0.02 | 0.02 | 0.02 | 0.05 |
| Comparative Example 1 | 0.02 | 0.02 | 0.02 | 0.05 |
| Comparative Example 2 | 0.01 | 0.02 | 0.04 | 0.08 |
| Comparative Example 3 | 0.06 | 0.05 | 0.03 | 0.10 |
| Comparative Example 4 | 0.00 | 0.00 | 0.00 | 0.01 |

It can be therefore said the invented catalyst would have sufficient ester conversion to target product during plant operation with mild reaction conditions such as medium pressures and temperatures, which means a considerable cost saving and lower risk.

1: A shaped catalyst body containing copper, aluminum, and manganese, wherein the shaped catalyst body has a packed bulk density of 0.87 to 1.43 g/cc.

2: The shaped catalyst body according to claim 1, wherein the shaped catalyst body comprises from 30% to 75% by weight of Cu, calculated as CuO.

3: The shaped catalyst body according to claim 1, wherein the shaped catalyst body comprises from 10% to 50% by weight of Al, calculated as Al₂O₃.

4: The shaped catalyst body according to claim 1, wherein the shaped catalyst body comprises from 1% to 25% by weight of Mn, calculated as MnO₂.

5: The shaped catalyst body according to claim 1, wherein the shaped catalyst body has a packed bulk density of 0.90 to 1.42 g/cc.

6: The shaped catalyst body according to claim 1, wherein the shaped catalyst body has a pore volume of 0.1 to 0.5 ml/g.

7: The shaped catalyst body according to claim 1, wherein the shaped catalyst body comprises from 40% to 65% by weight of Cu calculated as CuO.

8: The shaped catalyst body according to claim 1, wherein the shaped catalyst body comprises from 20% to 40% by weight of Al, calculated as Al₂O₃.

9: The shaped catalyst body according to claim 1, wherein the shaped catalyst body comprises from 5% to 20% by weight of Mn, calculated as MnO₂.

10: The shaped catalyst body according to claim 1, wherein the shaped catalyst body further comprises from 3% to 20% by weight of Zr, calculated as ZrO2.

11: The shaped catalyst body according to claim 1, wherein the shaped catalyst body has a bimodal pore size distribution.

12: The shaped catalyst body according to claim 11, wherein the shaped catalyst body exhibits a pore volume of 0.02 to 0.50 ml/g by pores with pore size in the range from 45 to 200 nm, and a pore volume of 0.20 to 0.60 ml/g by pores with pore size in the range from 10 to 200 nm.

13: The shaped catalyst body according to claim 11, wherein from 10% to 80% of the pore volume is formed by pores with pore size in the range from 45 to 200 nm, and from 70% to 100% of the pore volume is formed by pores with pore size in the range from 10 to 200 nm.

14: The shaped catalyst body according to claim 13, wherein from 35% to 70% of the pore volume is formed by pores with pore size in the range from 45 to 200 nm.

15: A process for producing the shaped catalyst body according to claim 1, comprising:
a) precipitation by combining an aqueous solution of a copper compound, a manganese compound, an aluminum compound and a precipitation agent;
b) filtration of the slurry and washing the precipitate;
c) drying and calcining at a temperature in a range from 200 to 1000° C. to form a thermally treated intermediate;
d) mixing the thermally treated intermediate; and
e) shaping to a shaped body.

16: A process for using the shaped catalyst body according to claim 1 for hydrogenation, dehydrogenation, hydrogenolysis, or ethynylation.
